(19) United States
(12) Patent Application Publication
(10) Pub. No.: US 2023/0410311 A1
(43) Pub. Date: Dec. 21, 2023

(54) CONTROL DEVICE, CONTROL METHOD, AND RECORDING MEDIUM

(71) Applicant: Konica Minolta, Inc., Tokyo (JP)

(72) Inventors: Akira HIROSHIGE, Tokyo (JP); Atsushi TANEDA, Tokyo (JP); Naoki HAYASHI, Tokyo (JP)

(21) Appl. No.: 18/328,910

(22) Filed: Jun. 5, 2023

(30) Foreign Application Priority Data
Jun. 17, 2022 (JP) 2022-098151

(51) Int. Cl. G06T 7/00

(52) U.S. Cl. CPC G06T 7/0016; G06T 2207/10124; G06T 2207/20081; G06T 2207/30004

(57) ABSTRACT

Disclosed is a control device, comprising: a hardware processor that acquires a radiographic image, wherein the hardware processor acquires an imaging condition of the radiographic image, automatically selects a type and a processing condition of predetermined processing to be executed on the acquired radiographic image based on the acquired imaging condition, and executes the selected predetermined processing on the radiographic image.

### CROSS REFERENCE TO RELATED APPLICATIONS

The present invention claims priority under 35 U.S.C. § 119 to Japanese Patent Application No. 2022-098151 filed on Jun. 17, 2022, the entire contents of which being incorporated herein by reference.

### TECHNICAL FIELD

The present invention relates to a control device, a control method, and a recording medium.

### DESCRIPTION OF THE RELATED ART

In the related art in a medical field, a radiographic imaging system executes predetermined processing on a captured image. For example, Japanese Unexamined Patent Publication No. 2016-87279 discloses that information indicating an elapsed period of time from a reference date and time to an imaging date and time is added to a captured image based on information on imaging of the subject.

Radiographic imaging requires a series of imaging tasks from positioning to imaging to checking an image and executing predetermined processing after the imaging.

A technician who performs radiographic imaging may handle a large number of imaging tasks a day. It is preferable to release a subject as soon as possible in order to reduce a physical burden on the subject during radiographic imaging. Therefore, the technician is required to execute and complete the imaging tasks in a short time.

The captured image is required to be an image easy for a clinician or a radiologist to examine, and it is important to appropriately execute predetermined processing called post-processing on the captured image.

### SUMMARY OF THE INVENTION

It is difficult to satisfy both doing a large number of imaging tasks and executing appropriate predetermined processing for obtaining an image easy to be diagnosed in a limited time of a day. Therefore, a burden on the technician increases, and a burden such as an increase in waiting time for imaging is imposed on patients and hospital operation.

In Japanese Unexamined Patent Publication No. 2016-87279, supplementary information indicating an elapsed time is added for progress observation. However, this is only associating one item of information in a specific case. Therefore, an improvement such as more efficiently executing processing in a large number of examinations that occur in one day cannot be expected, and reduction of the burden is insufficient.

An object of the present invention is to provide a control device, a control method, and a recording medium that can further reduce the workload of executing predetermined processing on a radiographic image.

To achieve at least one of the abovementioned objects, according to an aspect of the present invention, a control device reflecting one aspect of the present invention comprises:
a hardware processor that acquires a radiographic image, wherein
the hardware processor acquires an imaging condition of the radiographic image, automatically selects a type and a processing condition of predetermined processing to be executed on the acquired radiographic image based on the acquired imaging condition and executes the selected predetermined processing on the radiographic image.

To achieve at least another one of the abovementioned objects, according to an aspect of the present invention, a control method reflecting one aspect of the present invention comprises:
acquiring a radiographic image;
executing predetermined processing on the acquired radiographic image;
acquiring an imaging condition of the radiographic image; and
automatically selecting, based on the acquired imaging condition, a type and a processing condition of the predetermined processing to be executed, wherein
the executing includes executing the selected predetermined processing on the radiographic image.

To achieve at least another one of the abovementioned objects, according to an aspect of the present invention, a non-transitory computer readable recording medium reflecting one aspect of the present invention
causes a computer of a control device to function as:
a hardware processor that acquires a radiographic image, wherein
the hardware processor acquires an imaging condition of the radiographic image, automatically selects a type and a processing condition of predetermined processing to be executed on the acquired radiographic image based on the acquired imaging condition and executes the selected predetermined processing on the radiographic image.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention, wherein:

FIG. 1 is a diagram showing an X-ray imaging system according to an embodiment of the present invention.

FIG. 2 is a diagram illustrating the configuration of an imaging control device.

FIG. 3 is a flowchart showing the flow of imaging processing.

FIG. 4 is a view illustrating an example of an examination screen of the imaging control device.

FIG. 5 is a flowchart showing the flow of post-processing determination processing.

FIG. 6 is a view illustrating an example of the examination screen of the imaging control device.

FIG. 7 is a view illustrating an example of an examination screen of the imaging control device.

FIG. 8 is a view illustrating an example of an examination screen of the imaging control device.

FIG. 9A is a view illustrating an example of an examination screen of the imaging control device.

FIG. 9B is a view illustrating an example of an examination screen of the imaging control device.

FIG. 10 is a view illustrating an example of an examination screen of the imaging control device.

FIG. 11 is a view illustrating an example of an examination screen of the imaging control device.

FIG. 12 is a view illustrating comparison between current post-processing and previous post-processing.

FIG. 13 is a view illustrating the contents of current post-processing.

FIG. 14 is a view illustrating an example of an examination screen of the imaging control device.

FIG. 15 is a flowchart showing the flow of post-processing selection processing.

FIG. 16 is a view showing examples of the type of post-processing for each of specific imaging parts and specific cases.

FIG. 17 is a view showing an example of the examination screen of the imaging control device.

FIG. 18 is a view illustrating an example of the examination screen of the imaging control device.

### DETAILED DESCRIPTION

Hereinafter, embodiments of the present invention will be described in detail with reference to the drawings. However, the scope of the invention is not limited to the disclosed embodiments.

### First Embodiment

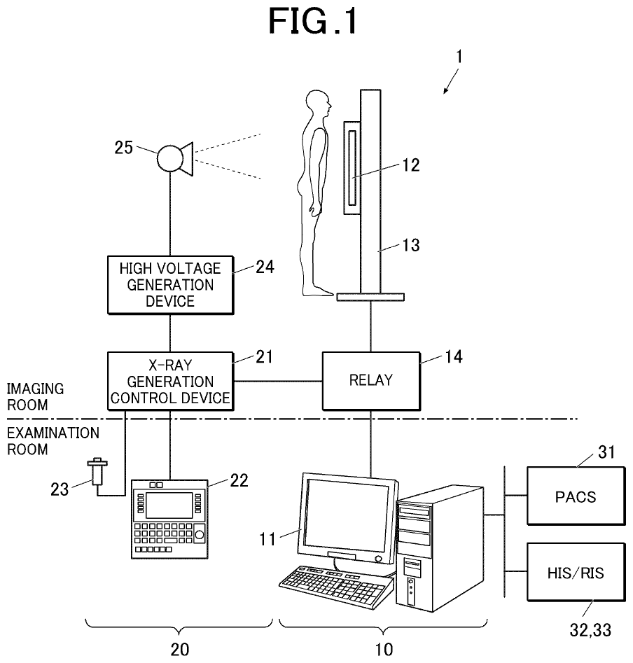

FIG. 1 is a diagram showing an X-ray imaging system 1 according to an embodiment of the present invention.

The X-ray imaging system 1 is an integrated imaging system in which an X-ray generation device 20 and an X-ray imaging device 10 exchange signals and the like with each other to perform X-ray (radiation) imaging in cooperation with each other. As used herein, X-ray (radiation) imaging will be referred to as imaging.

### Configuration of X-ray Imaging System

As illustrated in FIG. 1, the X-ray imaging system 1 includes the X-ray imaging device and the X-ray generation device 20.

The X-ray imaging system 1 is connected to a picture archiving and communication system (PACS) 31, a hospital information system (HIS) 32, and a radiology information system (RIS) 33 via a communication network.

The communication network, which includes the X-ray imaging system 1, the PACS31, the HIS32, and the RIS33, transmits and receives information according to, for example, the Digital Image and Communications in Medicine (DICOM) standard.

The X-ray imaging device 10 comprises an imaging control device 11 as a control device, a flat panel display (FPD) 12, an imaging table 13, a relay 14, and the like.

The X-ray imaging device 10 visualizes X-rays that have passed through an imaging target part such as the chest or the abdomen, for example, and captures an X-ray image that illustrates the state of the inside of the body. Hereinafter, the imaging target part is referred to as an imaging part. Hereinafter, an X-ray image is referred to as an image.

The FPD 12 is an imaging device that detects X-rays emitted from the X-ray tube device and passed through a subject.

For example, the FPD 12 is attached to the imaging table 13 and is connected to the imaging control device 11 through the imaging table 13 and the relay 14 by wired communication so as to communicate with each other.

The FPD 12 may be connected to the imaging control device 11 by wireless communication. In a case where the FPD 12 has the wireless communication function, the FPD 12 can be used with being placed on a bed on which the subject lies supine or with being held by the subject himself/herself instead of being mounted on a dedicated imaging stand 13.

The FPD 12 includes, for example, a scintillator, photo diodes (PD), and thin film transistor (TFT) switches arranged corresponding to the respective PDs (all not illustrated). The scintillator converts incident X-rays into light. The PDs are arranged in a matrix corresponding to pixels.

The FPD 12 converts incident X-rays into light by the scintillator, receives the light by the PDs, and accumulates the light as charges with respect to each pixel. The FPD 12 causes the charges accumulated in the PDs to flow out via the TFT switches and signal lines, and outputs the charges to the imaging control device 11 after amplification and A/D conversion.

The FPD 12 may be of the above-described indirect conversion type or may be of a direct conversion type that directly converts X-rays into electric signals.

The imaging stand 13 detachably holds the FPD 12 such that an X-ray incident surface of the FPD 12 faces the X-ray tube device 25. FIG. 1 illustrates an upright imaging stand for imaging a subject in an upright posture as an example of the imaging stand 13.

The imaging stand 13 may be a decubitus imaging stand for imaging a subject in a decubitus posture.

For example, the imaging stand 13 is communicably connected to the imaging control device 11 via the relay 14 by wired communication.

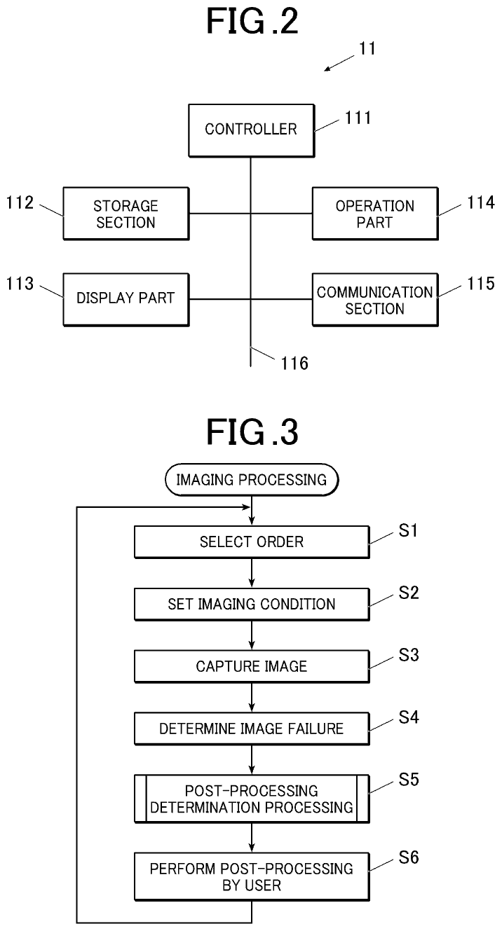

The imaging control device 11 controls the X-ray imaging system 1 in cooperation with the X-ray generation control device 21. For example, the imaging control device 11 transmits the detection conditions to the FPD 12 to set the conditions. The detection conditions include an image size to be captured, a frame rate (in the case of dynamic imaging), and information related to signal processing to be executed by the FPD 12. The information related to the signal processing performed by the signal processor FPD 12 is, for example, the gain of an amplifier, and the like. The imaging control device 11 controls the operations of the FPD 12, acquires an image from the FPD 12, performs predetermined image processing on the image, and displays the image on a display part 113 (see FIG. 2).

The imaging control device 11 may be a part of the X-ray generation device 20. For example, the imaging control device 11 can have a function as an X-ray generation console 22 of the X-ray generation device 20. This is a so-called integrated X-ray imaging system.

The X-ray imaging system 1 may include a display terminal device (not shown). The display terminal device may display the same content as that displayed on the display part 113 or a part of the content displayed on the display part 113. The display terminal device may have a function of image processing or a part of the functions of the X-ray generation console 22.

The X-ray generation device 20 includes an X-ray generation control device 21, the X-ray generation console 22, an irradiation switch 23, a high-voltage generation device 24, and the X-ray tube device 25.

The X-ray tube device 25 is placed at a position opposite to the FPD 12 across a subject. When the high-voltage generation device 24 applies a high voltage, the X-ray tube device 25 generates X-rays and irradiates a subject with the X-rays. The X-ray tube device 25 includes an X-ray movable diaphragm that adjusts an X-ray irradiation field.

The X-ray generation console 22 and the irradiation switch 23 are connected to the X-ray generation control device 21 via signal cables.

The X-ray generation console 22 is an operation table for inputting irradiation parameters and the like. The irradiation switch 23 is a switch for receiving an instruction of X-ray irradiation, and is constituted by, for example, a two-stage automatic return (momentary) push button switch. When the irradiation switch 23 is pressed to the first stage, the X-ray generation console 22 transmits a warm-up start signal for starting the warm-up of the X-ray tube device 25 to the X-ray generation control device 21. When the irradiation switch 23 is pressed to the second stage, the X-ray generation console 22 transmits an irradiation start signal for causing the X-ray tube device 25 to start X-ray irradiation to the X-ray generation control device 21.

The X-ray generation control device 21 controls the operations of the high-voltage generation device 24 and the X-ray tube device 25 based on irradiation parameters from the X-ray generation console 22 and the control signals from the irradiation switch 23. The irradiation parameter includes a radiation emission parameter. The control signals from the irradiation switch 23 include the warm-up start signal and the irradiation start signal.

The irradiation parameters can be set through the X-ray generation console 22 or can be set using the imaging control device 11.

FIG. 2 is a diagram illustrating the configuration of the imaging control device 11. As illustrated in FIG. 2, the imaging control device 11 includes a controller 111 (hardware processor), a storage section 112, a display part 113, an operation part 114, a communication section 115, and the like.

The controller 111 includes a central processing unit (CPU), a read only memory (ROM), a random access memory (RAM), and the like (none of which are illustrated).

In the ROM, basic programs and basic setting data are stored.

The CPU reads a program corresponding to the processing to be executed from the ROM or the storage section 112, develops the program in the RAM, and executes the developed program, thereby centrally controlling the operations of FPD 12 and the like.

The controller 111 acquires a plurality of radiographic images that satisfy a predetermined condition. The controller 111 functions as an acquirer.

The controller 111 specifies a radiographic image to be subjected to post-processing (predetermined processing) from among the plurality of radiographic images acquired by the acquirer. The controller 111 functions as a specifying section.

The controller 111 displays an identification to distinguish between the radiographic image on which the post-processing (predetermined processing) specified by the specifying section is to be performed and the radiographic image that is not specified by the specifying section. The controller 111 functions as a display controller.

The controller 111 executes post-processing (predetermined processing) on the radiographic image to be subjected to post-processing (predetermined processing) specified by the specifying section. The controller 111 functions as a processing section.

The controller 111 receives a user operation to execute the post-processing (predetermined processing) on the radiographic image to be subjected to the post-processing (predetermined processing) specified by the specifying section. The controller 111 functions as a receiving section.

The storage section 112 is, for example, an auxiliary storage device such as a hard disk drive (HDD) or a solid state drive (SSD). The storage section 112 may be a disk drive that reads and writes information by driving an optical disk such as a compact disc (CD) or a digital versatile disc (DVD), or a magneto-optical disk such as an MO disk. For example, the storage section 112 may be a memory card such as a USB memory or an SD card.

The storage section 112 stores various programs to be executed by the controller 111, parameters necessary for the execution of the programs, and data such as processing results.

The storage section 112 stores an image captured by the X-ray imaging device 10.

The storage section 112 stores examination order information.

The examination order information includes patient information on a patient as a subject, imaging conditions, examination items, an examination history of the subject, and request information.

The patient information is, for example, a patient ID, a patient name, a date of birth, a gender, and a location of the patient.

The imaging conditions include posture information at the time of imaging (for example, posture (standing position/lying position)), an irradiation direction (back/front/side), imaging part information (for example, chest), a tube voltage and a tube current, an irradiation time (mAs value), a frame rate (in the case of dynamic imaging), a physique of the subject, presence or absence of grid, and the like.

The examination items include lung ventilation function, pulmonary blood flow, or the like.

The examination history of the subject includes an imaging condition in the previous examination, or the like.

The request information includes a requesting department, an attending doctor, and a request comment.

The display part 113 includes, for example, a flat panel display such as a liquid crystal display or an organic EL display.

The display part 113 displays the contents of the examination order and a captured image based on a display control signal from the controller 111.

The operation part 114 includes a keyboard having cursor keys, number input keys, various function keys and the like, and a pointing device such as a mouse.

The operation part 114 receives an operation signal input as a key operation or a mouse operation, and outputs the operation signal to the controller 111.

The display part 113 and the operation part 114 may be integrally configured as, for example, a flat panel display with a touch screen.

The communication section 115 is a communication interface such as a network interface card (NIC), a MOdulator-DEModulator (MODEM), or a universal serial bus (USB), for example.

The controller 111 transmits and receives various information to and from a device that is connected to a network such as a wired/wireless LAN, according to the DICOM standard via the communication section 115.

A communication interface for short-range wireless communication such as near field communication (NFC) or Bluetooth (registered trademark) may also be applied as the communication section 115.

### Operation of X-ray Imaging System

Next, imaging processing in the X-ray imaging system 1 illustrated in FIG. 3 will be described.

It is assumed that an examination order is input in advance in the imaging control device 11. The examination order may be input from an external system such as an HIS32 or a RIS33 or may be manually input through the operation part 114 by a radiographer as a user. The radiographer is a technician, an image interpretation doctor, a diagnosing doctor, or the like.

### Imaging Processing

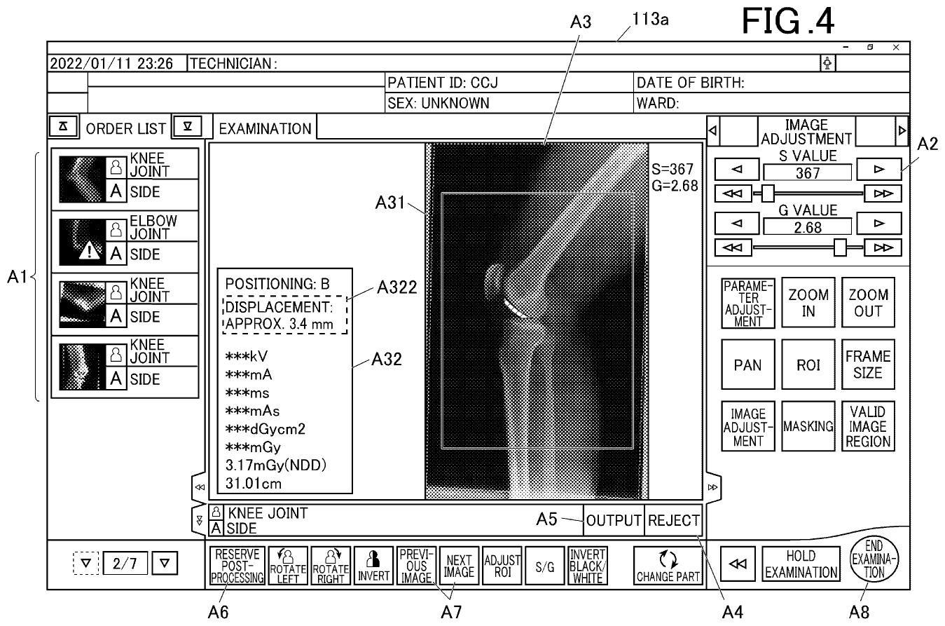

In the imaging processing, the controller 111 of the imaging control device 11 displays an examination screen 113a as illustrated in FIG. 4 on the display part 113. The controller 111 receives a selection of an examination order for which imaging is to be performed by a radiographer (step 51). The radiographer presses an imaging selection button A1 to select an examination order for which imaging is to be performed from among the saved examination orders.

FIG. 4 illustrates an example of an examination screen 113a displayed on the display part 113.

The controller 111 creates, in the examination screen 113a, imaging selection buttons A1 on which the contents (imaging part, imaging direction, and the like) of each imaging included in the examination order information are displayed. The controller 111 creates, in the examination screen 113a, a setting area A2 for image adjustment of the selected imaging and an image display area A3 for displaying a captured image. The controller 111 creates, in the examination screen 113a, an image failure (reject) button A4, an output button A5, and a post-processing reservation button A6 for setting an image as a post-processing target. The controller 111 creates, in the examination screen 113a, a switching button A7 for switching the image displayed in the image display area A3, an examination end button A8, and the like. In step S1, any image has not been displayed in the image display area A3 yet.

In response to an examination order being selected, the controller 111 transmits imaging conditions included in the examination order to the X-ray generation control device 21 and the FPD 12 and sets the imaging conditions therein (step S2).

Meanwhile, the radiographer places the subject between the X-ray tube device 25 and the imaging stand 13 and performs positioning. The positioning refers to, for example, a manner of the body position of the subject during imaging. The radiographer instructs the subject, for example, to take a certain breathing state (deep breathing or the like).

Next, the controller 111 receives a pressing operation of the irradiation switch 23 by the radiographer and performs imaging (step S3).

When the irradiation time is set as in simple X-ray imaging, X-ray irradiation is stopped when a predetermined irradiation time has elapsed. In the case of dynamic imaging, X-ray irradiation is continuously performed while the irradiation switch 23 is being pressed to the second stage. The X-ray irradiation is stopped when the irradiation switch 23 is released from being pressed.

The FPD 12 transmits the captured image to the imaging control device 11. Assuming that the radiographer has performed the above-described imaging plural times. That is, the controller 111 acquires a plurality of radiographic images captured by the same radiographer. The plurality of radiographic images captured by the same radiographer is a plurality of radiographic images that satisfy a predetermined condition.

The plurality of radiographic images satisfying the predetermined condition may be a plurality of radiographic images associated with the same examination order, a plurality of radiographic images of the same subject, or a plurality of radiographic images captured on the same imaging date. The plurality of radiographic images satisfying the predetermined condition may be a plurality of radiographic images of the same imaging part or a plurality of radiographic images captured within a predetermined period of time. The plurality of radiographic images satisfying the predetermined condition may be a plurality of radiographic images captured in one examination. In the plurality of radiographic images, the radiographer, imaging part, and requesting department may be different. The plurality of radiographic images may satisfy two or more conditions among same subject, same imaging date, same imaging part, same radiographer, and imaging within a predetermined period of time. The plurality of radiographic images satisfying the predetermined condition may be a plurality of frame images captured by single dynamic imaging. The plurality of radiographic images satisfying the predetermined condition may be a plurality of frame images captured by a plurality of dynamic imaging in one examination.

As shown in FIG. 4, the controller 111 displays the acquired image A31 and the imaging condition A32 of the imaging thereof in the image display area A3.

Next, the controller 111 receives a determination (image failure determination) by the radiographer as to whether or not the image displayed in the image display area A3 is to be rejected (step S4). To determine the image as a failure image, the radiographer presses an imaging failure button A4.

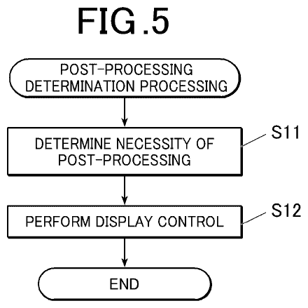

Next, the controller 111 determines whether or not it is necessary to perform post-processing as the predetermined processing on the image (step S5). The determination as to whether or not the post-processing is necessary corresponds to post-processing determination processing illustrated in FIG. 5.

The post-processing includes at least one of ROI (Region Of Interest) adjustment, valid image region setting, S/G value adjustment, rotation/inversion/arbitrary angle rotation, image processing (E processing/F processing/H processing/scattered radiation correction/image processing condition change, etc), enhancement processing (frequency enhancement processing for catheter tip enhancement processing, gauze enhancement processing/other enhancement processing, etc), grid removal processing, masking, trimming, marker/stamp/overlay, and output setting.

### Post-Processing Determination Processing

In the post-processing determination processing, the controller 111 determines whether or not it is necessary to perform post-processing on the image captured in step S3 (currently captured image). The controller 111 stores the result of the determination in the storage section 112 (step S11). That is, the controller 111 specifies a radiographic image on which the predetermined processing (post-processing) is to be performed, from among the plurality of radiographic images acquired by the controller 111 as the acquirer. When the current imaging is dynamic imaging, the controller 111 may make the determination on a frame image basis or may make the determination on a plurality of frame images collectively as one imaging.

Specifically, the controller 111 compares the currently captured image with a preset image for comparison, and determines, based on the result of the comparison, whether or not the post-processing is necessary.

For example, a case will be described where the image for comparison is a previous image obtained by imaging the same imaging part as the current imaging part. In this case, when there is no difference in imaging conditions, alignment, resolution, or the like between the currently captured image and the previously captured image, the controller 111 determines that the post-processing is unnecessary.

The case will be described where the controller 111 compares the currently captured image with the previously captured image of the same imaging part and determines that there is the above-described difference. In this case, as illustrated in FIG. 4, the controller 111 displays a comparison result A322 indicating the difference in the image display area A3. The comparison result A322 is supplementary information for indicating that the post-processing is necessary, or for performing focused confirmation and adjustment in the post-processing. Therefore, the comparison result A322 may not only indicate the coordinate difference of a specific structure but may also indicate how much the difference should be corrected based on a threshold value of an acceptable difference.

The controller 111 determines the necessity of performing the post-processing on the currently captured image based on the post-processing performed on the preset image for comparison.

For example, a case will be described where the image for comparison is a previously captured image of the same imaging part as the current imaging part, and the post-processing performed on the previous image is only predetermined fixed processing. In this case, the controller 111 automatically performs the same fixed processing on the currently captured image and determines that the other post-processing is unnecessary. The fixed processing is preset basic processing. Specifically, the fixed processing includes addition of an annotation indicating a horizontal direction or application/non-application of scattered radiation correction processing according to use/non-use of a grid at the time of imaging. In the case of follow-up observation, it is often desired to interpret the image in the same size as the previous image. Therefore, the fixed processing in this case may include only output processing for storage or the like and may not include automatic adjustment by trimming and image processing for a specific modality. When the currently captured image meets a specific condition, the controller 111 determines that the post-processing for the currently captured image is unnecessary. The specific condition is a specific imaging part, a specific use, a respiratory cycle, or the like.

For example, a case will be described where the imaging part of the currently captured image is the front/side of the chest which is frequently captured part in general imaging. In this case, the controller 111 determines that the post-processing is unnecessary because the image processing condition is often fixed as a routine.

In a case where the imaging part is an orthopedic joint such as a knee joint, the viewability of the region of interest often changes depending on the irradiation direction or the angle of the subject, and the post-processing often improves the viewability. Therefore, in this case, the controller 111 determines that the post-processing is necessary.

In a case where the imaging part is the front/side of the chest and a specific department or a specific doctor requests the imaging, the controller 111 considers the preference of the doctor regarding the gradation processing degree and determines that the post-processing is necessary. In one example, the controller 111 makes the determination based on the imaging part but may make the determination as to whether or not the post-processing is necessary in combination with another condition.

For example, when the currently captured image is used for surgery as a specific usage, the controller 111 determines that the post-processing for the currently captured image is unnecessary because the captured image is immediately checked.

The controller 111 determines the necessity of performing post-processing on the currently captured image based on an input operation by the user. The user as used herein may also be a technician, an image interpretation doctor, a diagnosing doctor, or the like other than the radiographer.

To be specific, the controller 111 receives a user determination as to whether or not to perform post-processing on the currently captured image, through a post-processing reservation button A6. When the user determines to perform the post-processing on the currently captured image, the user presses the post-processing reservation button A6. When the post-processing reservation button A6 is pressed, the controller 111 determines that post-processing is necessary.

The controller 111 determines the necessity of post-processing on the currently captured image based on external information.

For example, a case will be described where the X-ray imaging system 1 comprises an optical camera (not illustrated) that captures an optical image of an area including at least a part of an area irradiated with X-rays from the X-ray tube device 25. That is, the optical camera may capture an image of an area including the entire subject or the X-ray imaging device 10, and the captured area is not limited to the region irradiated with X-rays.

In this case, the controller 111 determines the imaging direction of the currently captured image based on the optical image as the external information. The controller 111 performs processing of adding the determined imaging direction as an annotation to the currently captured image. The controller 111 determines that this processing is unnecessary in the post-processing.

For example, the controller 111 may detect a foreign object such as gauze based on the optical image as the external information. When a foreign object such as gauze is not detected, the controller 111 determines that the catheter tip enhancement processing and the gauze enhancement processing are unnecessary in the post-processing.

For example, the controller 111 may determine whether or not a grid was used in the current imaging based on the optical image as the external information. When the grid is not used, the controller 111 determines that grid removal processing is unnecessary in the post-processing.

For example, the controller 111 determines the posture of the subject at the time of imaging based on the optical image as the external information. The controller 111 performs processing of adding the determined posture of the subject to the currently captured image as an annotation or comment. The controller 111 determines that this processing is unnecessary in the post-processing.

For example, the controller 111 determines the posture of the subject at the time of imaging based on the optical image as the external information. The controller 111 determines whether or not the post-processing is necessary based on the subject and X-ray irradiation position or the X-ray irradiation direction. The X-ray irradiation direction is, for example, irradiation from the back of the subject or irradiation from the front of the subject. For example, when the imaging part is the ankle, the controller 111 determines the posture of the subject on the imaging table, i.e., whether his/her foot is in a standing or lying position. The controller 111 further determines whether the posture is the same as the posture of the subject at the time of the previous imaging. The controller 111 determines whether or not post-processing is necessary based on the determination result.

For example, the controller 111 determines, based on the optical image as the external information, whether imaging has been performed with the subject sitting or standing. The controller 111 determines the load on the foot of the subject and the gravity direction based on the determination result.

When the controller 111 determines the posture of the subject at the time of imaging, an optical image of the entire subject may be used as the external information.

Next, the controller 111 performs display control of the examination screen 113a so that the user can distinguish between the image for which post-processing is determined necessary in step S11 and the image for which post-processing is determined unnecessary (step S12) and ends the processing.

Specifically, the controller 111 attaches an identification mark or the like to the image that has been determined to require post-processing.

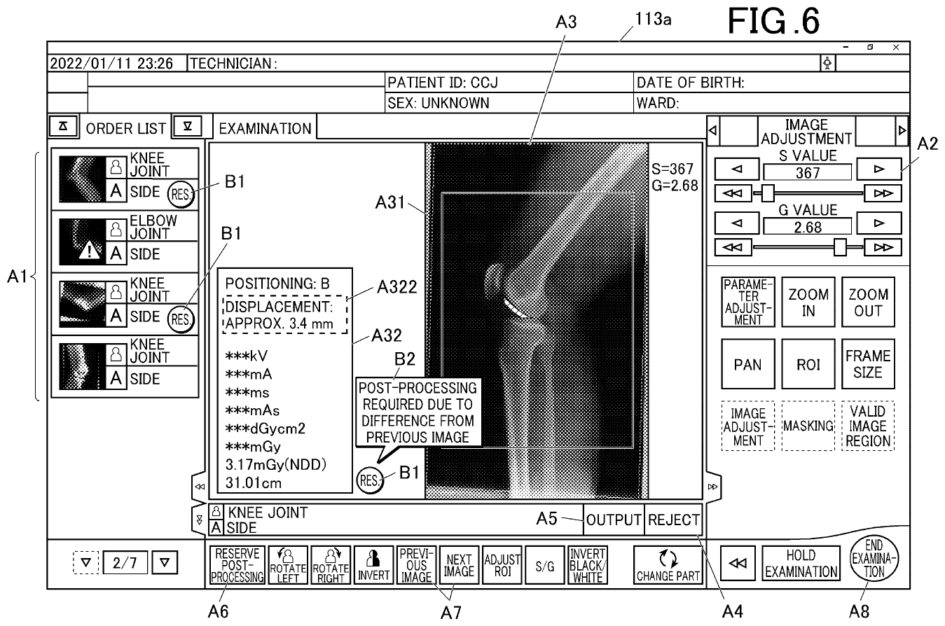

For example, as illustrated in FIG. 6, the controller 111 adds a post-processing reservation mark B1 as the identification mark to the image for which the post-processing has been determined necessary.

The controller 111 may add to the image for which post-processing has been determined unnecessary, an identification mark or the like indicating that post-processing is unnecessary.

The controller 111 may add a character or a symbol to the image instead of the identification mark.

The controller 111 may display the identification mark or the identification character or symbol on any of the imaging selection button A1, a thumbnail in the imaging selection button A1, and a dialog instead on the image. A case will be described where single imaging corresponding to a single imaging selection button A1 produces a plurality of images. In this case, when the controller 111 determines that post-processing is necessary for at least one of the plurality of images, the controller 111 adds an identification mark or a character or symbol for identification to the imaging selection button A1.

The controller 111 may change the color or shape of the imaging selection buttons A1 so that the user can distinguish between the image for which post-processing is determined necessary and the image for which post-processing is determined unnecessary.

In an example shown in FIG. 6, a plurality of examination orders for one patient having a patient ID “ccj” is shown. Although identification marks are added to the images that requires post-processing among the images captured according to the examination order, the display manner is not limited thereto. On an examination list screen (not illustrated) on which each examination order is displayed in one line, an identification mark or the like may be added to an examination order that includes an image requiring post-processing.

As shown in FIG. 6, the controller 111 may display a reason B2 for determining whether or not it is necessary to perform the post-processing in step S11, on the currently captured image in the examination screen 113a as an overlay, a comment, or the like.

The controller 111 may allow only the image for which post-processing is determined necessary to be displayed in the image display area A3.

For example, when the user switches the image displayed in the image display area A3 with the switching button A7, the controller 111 excludes the image for which post-processing is determined unnecessary from the images (switching targets) switchable with the switching button A7. When a predetermined imaging is selected from the examination order list by using the imaging selection button A1, the controller 111 may display the image corresponding to the selected imaging in the image display area A3 even when the image is not a switching target.

A case will be described where the current imaging is dynamic imaging, and the controller 111 determines necessity of post-processing with respect to each frame image and displays only the frame images for which the post-processing has been determined necessary on the display part 113. This is, for example, a case where the range to be examined can be specified from among all frame images of one photographing. The range to be examined is a range to be provided to a clinician or the like. The case where the range to be examined can be specified is, for example, a case where the range to be examined can be limited to one cycle of breathing or the like.

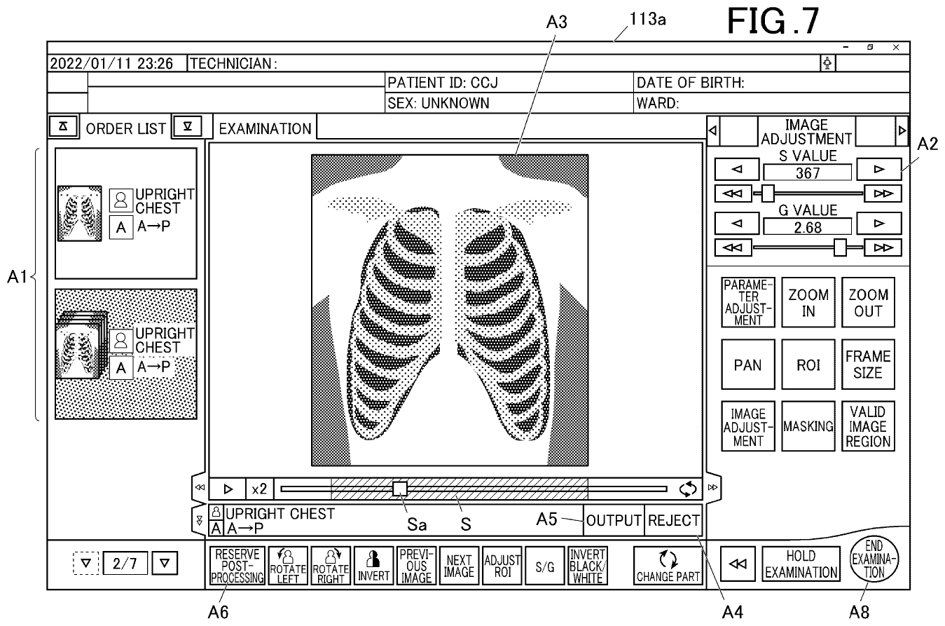

In this case, as illustrated in FIG. 7, the controller 111 creates a seek bar (replay bar) S in a lower part of the examination screen 113a. The slider Sa displayed on the seek bar S indicates the position of the frame image currently displayed in the image display area A3 in the entire dynamic image.

In the seek bar S, the controller 111 displays the range of the frame images for which post-processing has been determined necessary and the range of the frame images for which post-processing has been determined unnecessary in different colors. In the example shown in FIG. 7, the range of frame images for which post-processing has been determined necessary is displayed by hatching, and the range of frame images for which post-processing has been determined unnecessary is displayed in white. The slider Sa is movable only within the range indicated by the hatching. The range indicated by the hatching is the range of frame images for which the post-processing has been determined necessary.

A case will be described where the current imaging is dynamic imaging and the frame image displayed in the image display area A3 is switchable by using the switching button A7. In this case, the controller 111 switches the frame images within the range where post-processing has been determined necessary. That is, the controller 111 exclude the frame images for which the post-processing has been determined unnecessary from the targets switchable by the switching button A7 (switching targets).

A case will be described where the current imaging is dynamic imaging and the controller 111 makes a determination as to whether or not post-processing is necessary with respect to each frame image, and as a result, the controller 111 determines that all frame images in one imaging are to be subject to post-processing. This is, for example, a case where one imaging does not cover one cycle of breathing, or a case where time series or difference processing is required using frame images before and after the frame image for which post-processing has been determined necessary. Specifically, in a case where tone processing is required for a frame image with maximum inhalation, tone matching is required for the previous and subsequent frame images if the frame images are viewed as a moving image. Therefore, post-processing is also required for frame images other than the frame image for which post-processing has been determined necessary. As a result, post-processing may sometimes be required for all of the frame images. In this case, the controller 111 sets that all of the frame images are viewable on the display part 113.

In such cases as described above, a case will be described where the purpose of examination or analysis can be confirmed in advance from the examination order information or the like. In this case, the controller 111 determines whether it is possible to specify the range of the frame images for which post-processing is necessary according to the examination purpose or the analysis purpose. If it is possible to specify the range of the frame images, the controller 111 may display only the frame images requiring the post-processing on the display part 113 as shown in FIG. 7.

The controller 111 performs display control of the examination screen 113a so that the processing determined unnecessary as the post-processing in the step S11 cannot be performed.

For example, as illustrated in FIG. 6, the controller 111 grays out (shown as broken lines in FIG. 6) some of the buttons in the setting area A2 so that they cannot be selected. The controller 111 may not display, on the examination screen 113a, a button for selecting processing that has been determined unnecessary as the post-processing. The controller 111 may display, on the examination screen 113a, only a button for selecting processing that has been determined necessary as the post-processing.

The controller 111 may set, in step S11, the degree of necessity (priority) of post-processing with respect to an image for which the post-processing has been determined necessary. The priority is, for example, “post-processing is necessary”, “it is better to perform post-processing”, “it is better to check the necessity of post-processing”, or the like. The priority may be input by a user through the operation part 114 or may be set by the controller 111.

The controller 111 performs, in step S12, display control in accordance with the priority set in step S11. More specifically, the controller 111 adds a mark or a text according to the degree of priority to the image or changes the color of the image so that the priority is recognizable. The text is, for example, “check required” and “precaution”.

The controller 111 may perform, in step S12, display control according to the processing that has been determined necessary among a variety of post-processing in step S11. Such processing is, for example, gradation adjustment, review of the output destination, readjustment of the masked area, or the like.

Specifically, the controller 111 adds a mark or a text to the image or changes the color of the image corresponding to the processing determined necessary so that the difference in the processing is recognizable.

The controller 111 may display, in the examination screen 113a, a comment or the like according to the processing that has been determined necessary. The comment may be input by a user through the operation part 114 or may be set by the controller 111.

Referring back to FIG. 3, when the post-processing determination processing ends, the radiographer releases the subject from the positioning.

Next, the controller 111 receives a user operation of post-processing on the image for which the post-processing has been determined necessary in step S5 (step S6). Thereafter, the controller 111 continues the processing with step S1 to proceed to the next imaging. That is, the controller 111 receives a user operation to execute predetermined processing (post-processing) on the radiographic image specified by the controller 111 itself as the specifying section. The user as used herein may also be a technician, an image interpretation doctor, a diagnosing doctor, or the like other than the radiographer. The user who performs imaging, makes image failure determination and determination as to whether or not post-processing is necessary is different from the user who performs post-processing. This allows the post-processing user to perform the post-processing for multiple examinations at once in a terminal device that can communicate with the imaging control device 11. The user who performs imaging and makes image failure determination, the user who determines whether or not post-processing is necessary, and the user who performs post-processing may be different one another.

In step S6, the controller 111 stores the result of performing the post-processing (whether or not the post-processing has been performed) on the image in the storage section 112.

In step S6, the controller 111 itself may perform the post-processing on the image that has been determined to require post-processing in step S5. That is, the controller 111 execute the predetermined processing (post-processing) on the radiographic image specified by the controller 111 itself as the specifying section.

An example will be described in which the controller 111 itself performs the post-processing on the image for which post-processing has been determined necessary in step S5.

For example, the controller 111 performs the fixed processing applied to the previous image obtained by imaging the same imaging part as the current imaging part on the image for which post-processing has been determined necessary. The fixed processing is gradation processing, trimming, or the like.

The controller 111 adds the posture information of the subject determined based on the optical image as an annotation to the image for which post-processing has been determined necessary.

A case will be described where the controller 111 determines, based on the patient information, that the location of the patient is an operating room. In this case, the controller 111 applies gauze enhancement processing or the like to the image for which post-processing has been determined necessary, so as to generate a gauze-enhanced image.

In a case where the location of the patient is an intensive care unit (ICU) or a neonatal intensive care unit (NICU), the patient needs to be withdrawn soon. Therefore, there is a possibility that it cannot spend long time to perform post-processing after imaging. A case will be described where the controller 111 determines that the location of the patient is an ICU or NICU based on the patient information. In this case, the controller 111 performs the post-processing that requires a short processing time or the post-processing that is a relatively light load, on the image determined to require post-processing. For example, when the currently captured image is a still image, the controller 111 performs post-processing on the image determined to require post-processing because the processing load of post-processing on the still image is relatively light. In contrast, when the current imaging is dynamic imaging, the processing load of post-processing on the dynamic image is heavy in comparison with post-processing on a still image since a dynamic image includes a plurality of frame images. Therefore, the controller 111 causes the display part 113 to display the original currently captured image without post-processing.

The controller 111 may preset the allowable range of the processing time or the type of the post-processing which the controller 111 automatically performs on the image determined to require the post-processing with respect to each facility or environment.

A case will be described in which, in step S6, the controller 111 automatically performs post-processing on the image determined to require post-processing in step S5, while the user does not perform post-processing. In this case, when the examination end button A8 is pressed, the controller 111 may display a warning dialog on the examination screen 113a. With the warning dialog, the controller 111 informs that the post-processing has been automatically performed but the post-processing by the user has not been performed.

A case will be described where, in step S6, the examination end button A8 is pressed by the user without the controller 111 and the user performing post-processing on the image that has been determined to require post-processing in step S5. In this case, the controller 111 may display, on the examination screen 113a, a warning dialog indicating that post-processing has not been performed.

### Post-Post-Processing Display Processing

Next, display processing after performing post-processing will be described, which is for controlling the display of an image on which the user has performed post-processing and an image on which the user has not performed post-processing in step S6 of the imaging processing.

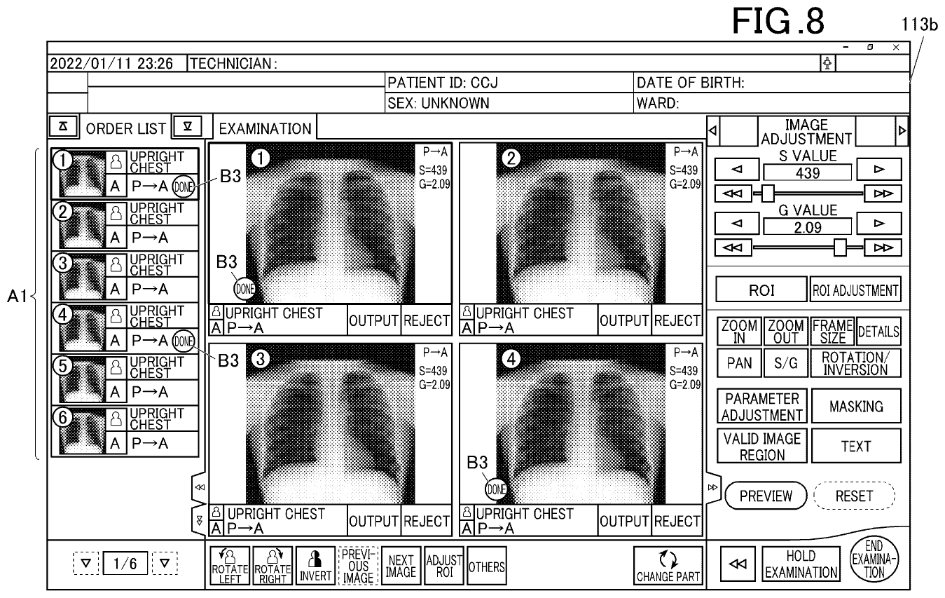

FIG. 8 illustrates an example of the examination screen 113b after execution of the post-processing in the above-described step S6.

In the example shown in FIG. 8, the numbers on the imaging selection buttons A1 correspond to the numbers on the images displayed in the image display area A3.

In the post-post-processing display processing, the controller 111 performs display control of the examination screen 113b so that the user can distinguish between an image on which post-processing has been performed and an image on which post-processing has not been performed.

Specifically, the controller 111 attaches an identification mark or the like to the image on which the user has performed the post-processing.

For example, as illustrated in FIG. 8, the controller 111 adds done marks B3 as identification marks to the imaging selection buttons A1 corresponding to images on which the user have performed post-processing and to the images on which the post-processing have been performed.

The controller 111 may add an identification mark or the like indicating that post-processing has not been performed, to the imaging selection button A1 corresponding to the image on which the user has not performed post-processing and to the image on which post-processing has not been performed.

The controller 111 may add a character or a symbol to the imaging selection button A1 and to the image instead of the identification mark.

The controller 111 may display an identification mark or an identification character or symbol on any of the thumbnail or the dialog in the imaging selection button A1 in addition to on the imaging selection button A1 and the image. A case will be described in which one imaging corresponding to one imaging selection button A1 includes a plurality of images and a user has performed post-processing on at least one of the plurality of images. In this case, the controller 111 adds an identification mark or an identification character or symbol to the imaging selection button A1.

The controller 111 may change the color, shape or the like of the imaging selection button A1 so that the user can distinguish an image on which post-processing has been performed from an image on which post-processing has not been performed.

The ON/OFF of the identification, the way of displaying the identification, the screen configuration of the examination screen 113b, and the like in the post-post-processing display processing may be set by the user through an input operation. The ON/OFF of the identification, the way of displaying the identification, the screen configuration of the examination screen 113b, and the like may be set in advance with respect to each facility, each engineer, each examination type, or each department.

With the post-post-processing display processing described above, the user can easily distinguish between an image on which the user has performed post-processing and an image on which the user has not performed post-processing. This allows the user to check whether post-processing is certainly unnecessary for an image on which post-processing has not been performed. The user can check whether the post-processing that has already been performed on the image is appropriate.

In the post-post-processing display processing, the controller 111 may display an image on which the user has performed post-processing and an unprocessed image (original image) thereof side by side on the examination screen 113b.

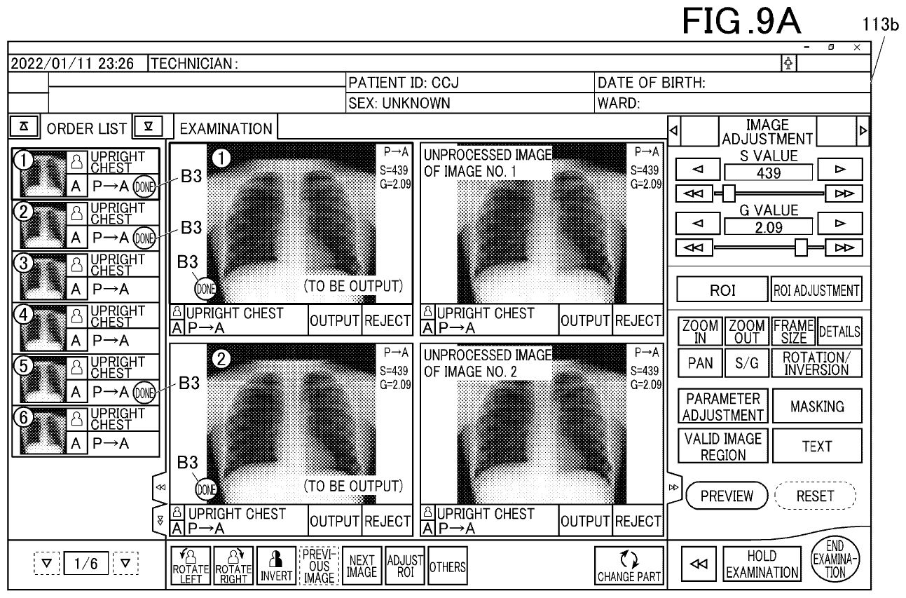

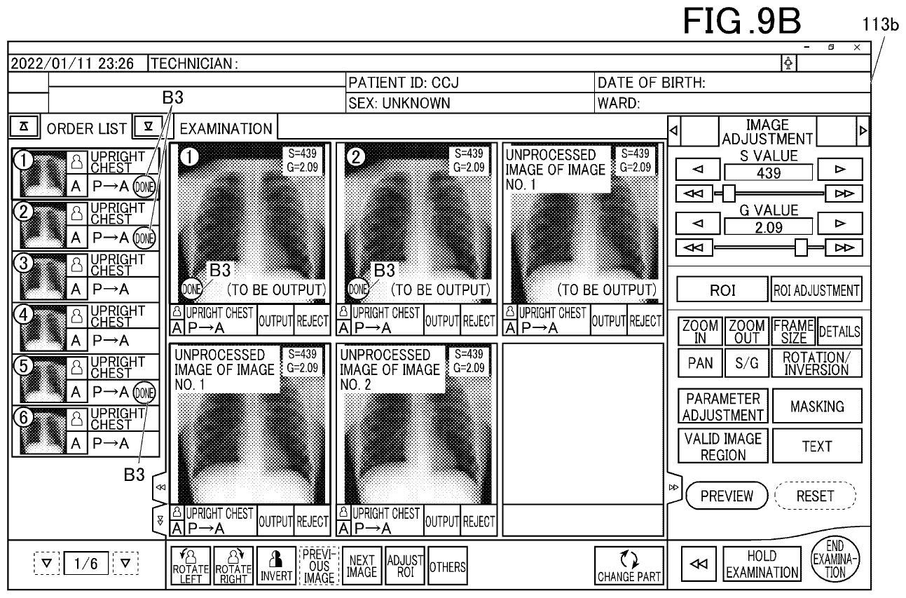

FIG. 9A and FIG. 9B illustrate examples of the examination screen 113b in which images on which post-processing has been performed by the user and the original images thereof are displayed side by side in the image display area A3.

In the example shown in FIG. 9A, the controller 111 displays images on which post-processing has been performed by the user and the original images thereof right and left in the image display area A3. The controller 111 displays the images to be output on the left side of the image display area A3.

In the example shown in FIG. 9B, the controller 111 displays images on which post-processing has been performed by the user and the original images thereof vertically side by side in the image display area A3. As for the image on which image processing has not been performed, the controller 111 displays only the unprocessed image, such as the image No. 3 shown in FIG. 9B. The controller 111 displays the images to be output in an upper part of the image display area A3.

With the above display, the user can compare the images on which post-processing has been performed by the user with the unprocessed images (original images) thereof to check whether the applied post-processing is appropriate.

The controller 111 may display only the images on which post-processing has been performed by the user in the image display area A3.

The controller 111 may display images on which the user has performed post-processing and the original images thereof side by side in another dialog or screen instead of the image display area A3.

In the post-post-processing display processing, the controller 111 may display, on the examination screen 113b, an image on which the user has performed post-processing and a temporarily generated image generated from the captured image in the steps of the post-processing. The temporarily generated image is, for example, a pre-stitched images for a long image, a rejected image, or a predetermined frequency enhanced image. The predetermined frequency enhanced image is, for example, a catheter tip enhanced image or a gauze enhanced image.

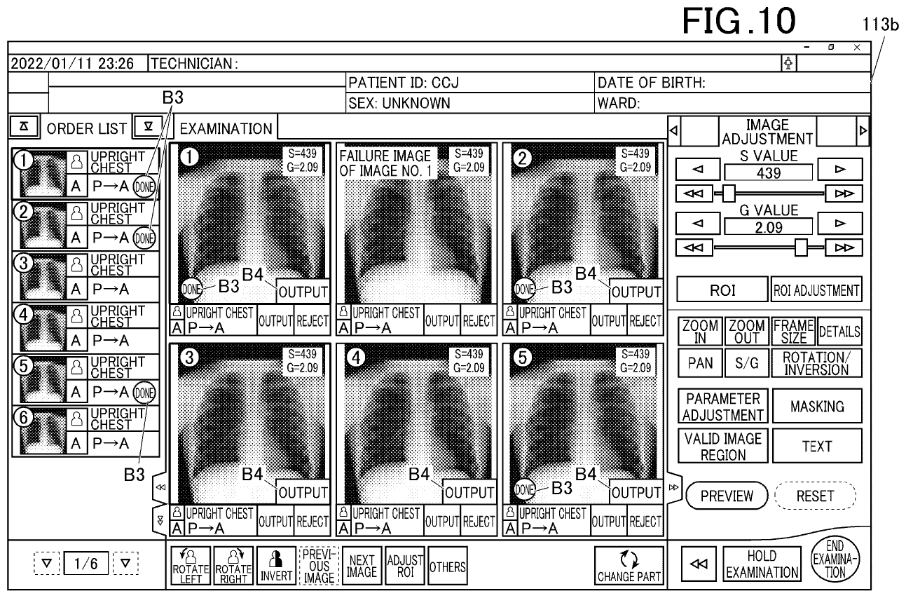

FIG. 10 illustrates an example of the examination screen 113b in which images on which the user has performed post-processing and a temporarily generated image (rejected image) in the image display area A3.

As illustrated in FIG. 10, the controller 111 adds identification information B4, which is a mark, a character, or the like to the image to be output. The identification information B4 is added to distinguish between the image to be output and an image not to be output. The identification information B4 may be shown as a frame of an image or a different color.

In a case where it is only intended to distinguish between the image to be output and the image not to be output, the done mark B3 may be omitted. However, it is preferable to add the done mark B3 to a temporarily generated image. This is because the user can distinguish between an image generated through post-processing by the user and an image generated through automatically performed post-processing (by the controller 111).

With the display, a user can confirm whether or not the temporarily generated image is to be output.

The controller 111 may display an image on which the user has performed post-processing and a temporarily generated image in another dialog or screen instead of the image display area A3.

A case will be described in which the number of images on which the user has performed post-processing and the number of temporarily generated images are large, and it is impossible to display all of the post-processed images and the temporarily generated images on the examination screen 113b. In this case, the controller 111 may create a slider bar on the examination screen 113b so that the displayed images are switchable. In this case, the controller 111 may increase the number of images that can be displayed on the examination screen 113b.

In the post-post-processing display processing, the controller 111 may display, on the examination screen 113b, an image on which the user has performed post-processing and a previous image corresponding to the image. The corresponding previous image is a previous image obtained by imaging the same imaging part as the imaging part of the image on which the user has performed the post-processing.

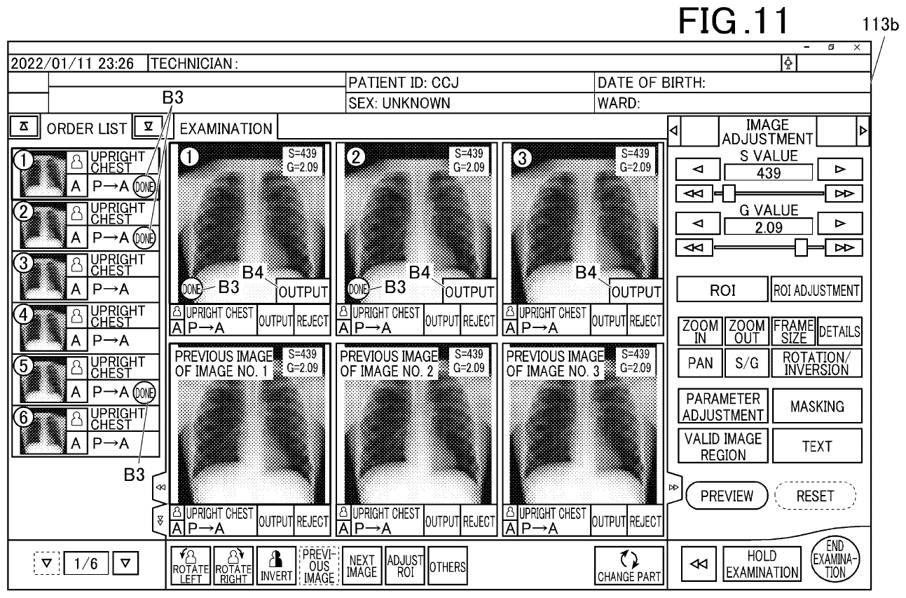

FIG. 11 illustrates an example of the examination screen 113b in which images on which the post-processing has been performed by the user and the corresponding previous images are displayed vertically side by side in the image display area A3. The controller 111 may display images on which post-processing has not been performed by the user and the corresponding previous images vertically side by side, such as the image No. 3 illustrated in FIG. 11. The controller 111 displays the images to be output in an upper part of the image display area A3.

With the display described above, the user can compare an image on which the post-processing has been performed by the user with the corresponding previous image and check whether the applied post-processing is appropriate again before outputting the image.

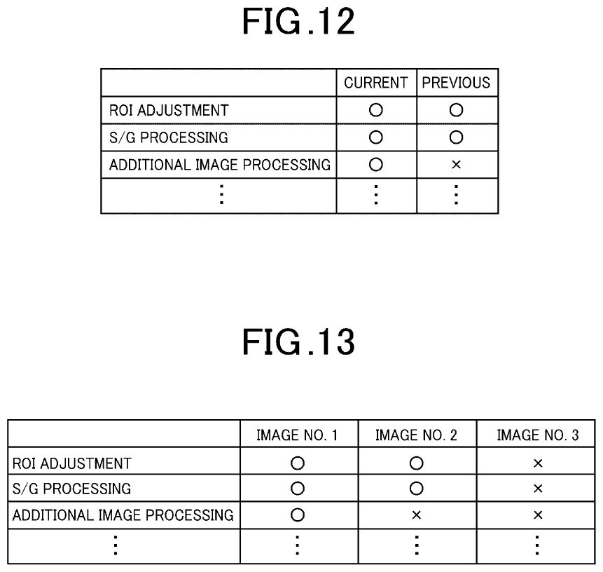

The controller 111 may display, on the examination screen 113b, information on whether or not post-processing has been performed on the previous image corresponding to the image post-processed by the user, or the contents of the post-processing (the type of the post-processing) that has been performed on the previous image. In this case, as shown in FIG. 12, the controller 111 may display a table for comparing the post-processing performed on the currently captured image with the post-processing performed on the previously captured image on the examination screen 113b. In the example illustrated in FIG. 12, the mark “o” (processed) and the mark “x” (unprocessed) indicate whether or not processing has been performed.

In the post-post-processing display processing, the controller 111 may display the contents of the post-processing performed by the user (the type of post-processing) in the examination screen 113b.

FIG. 13 is a table showing the contents of the post-processing performed by the user on images No. 1 to No. 3 to be output. In the table of contents of the post-processing, variables of the processing may be displayed instead of indicating whether or not the processing has been performed with circles and cross marks (○ and x).

When a predetermined image is selected by the user through an input operation on the examination screen 113b, the controller 111 may display the contents of the post-processing performed on the selected image in the setting area A2.

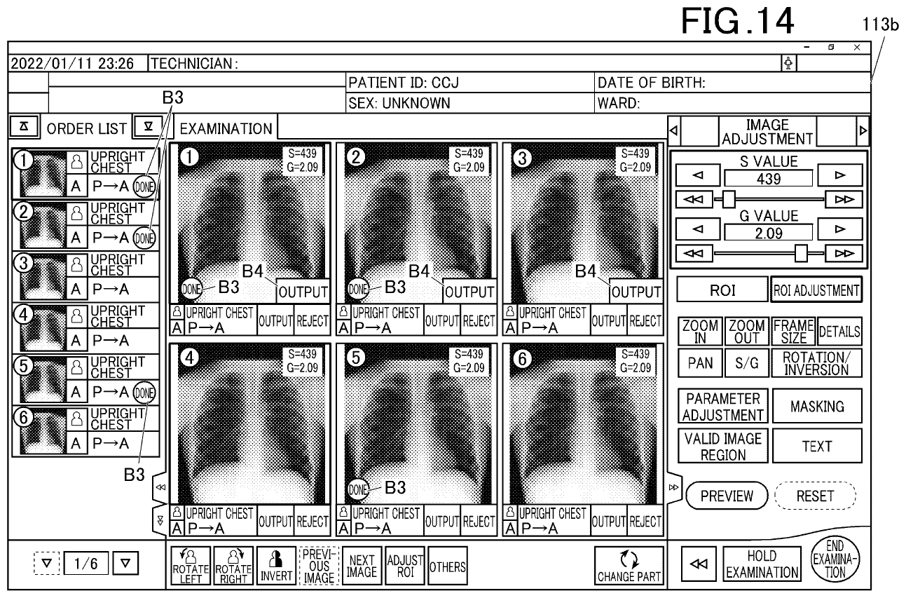

FIG. 14 illustrates an example of the examination screen 113b on which the contents of the post-processing is displayed in the setting area A2.

In the example illustrated in FIG. 14, the controller 111 adds a black frame to the button for selecting the processing that has been performed as the post-processing in the setting area A2. The controller 111 may change the color of the button for selecting the processing that has been performed as the post-processing.

With the display described above, the user can collectively check the contents of the post-processing that has been performed on the image to be output before outputting the image.

As described above, the controller 111 executes the post-post-processing display processing. As a result, the user can check the result of the post-processing by the user more efficiently while he/she can view a plurality of images at once. The user can efficiently make the final confirmation as to whether the image to be output to the PACS or the like is an image suitable for the examination order.

### Second Embodiment

Next, the X-ray imaging system 1 according to a second embodiment will be described. The following descriptions will be made focusing on the differences from the first embodiment.

In step S2 of the imaging processing in the X-ray imaging system 1 of the present embodiment, the controller 111 acquires, from the storage section 112, the imaging conditions included in the examination order selected by the radiographer. The controller 111 transmits the imaging conditions to the X-ray generation control device 21 and the FPD 12 to set the imaging conditions. That is, the controller 111 acquires the imaging conditions of the radiographic image. The controller 111 functions as a second acquirer.

Next, in step S3 of the imaging processing, the controller 111 performs imaging and acquires a captured image from the FPD 12. That is, the controller 111 acquires a radiographic image. The controller 111 functions as a first acquirer. The radiographic image may be a plurality of frame images captured by a single dynamic imaging.

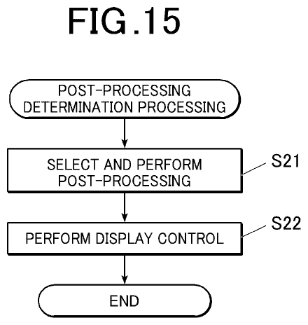

Next, in step S5 of the imaging processing, the controller 111 executes post-processing selection processing illustrated in FIG. 15 instead of the post-processing determination processing.

### Post-Processing Selection Processing

In the post-processing selection processing, the controller 111 selects, based on the imaging conditions, the type and processing conditions of the post-processing to be performed on the image captured in step S3 (currently captured image). The controller 111 performs the selected post-processing on the currently captured image (step S21). In a case where the current imaging is dynamic imaging, the controller 111 performs the processing of step S21 on a frame image basis.

That is, the controller 111 automatically selects, based on the imaging conditions acquired as the second acquirer, the type and processing conditions of the predetermined processing (post-processing) to be executed as a processing section (the controller 111). The controller 111 functions as a selection section.

The controller 111 executes the predetermined processing (post-processing) selected by the selection section. The controller 111 functions as a processing section.

The post-processing selection processing may be performed in parallel with the image failure determination in step S4 of the imaging processing at the same time.

The type of the post-processing is any one of region of interest (ROI) adjustment, valid image region setting, S/G value adjustment, rotation/inversion/arbitrary angle rotation, image processing (E processing/F processing/H processing/scattered ray correction/change of image processing conditions and the like), enhancement processing (catheter tip enhancement processing, gauze enhancement processing/other enhancement processing and the like, which are frequency enhancement processing), grid removal processing, masking, trimming, marker/stamp/overlay, and output setting.

The processing conditions of the post-processing are the parameters of each processing, for example, the frequency band to be enhanced in the frequency enhancement processing.

In step S21, the controller 111 selects the type and processing conditions of post-processing based on the post-processing performed on the previous image obtained by imaging the same imaging part as the current imaging part. The controller 111 performs the selected post-processing on the currently captured image.

Specifically, the controller 111 determines that the type and processing conditions of the post-processing that are the same as the type and processing conditions of the post-processing performed on the previous image are to be performed on the currently captured image.

For example, the controller 111 performs position adjustment and image processing on the currently captured image so that the valid image region and the ROI of the currently captured image become the same as those of the previous image.

When the currently captured image meets a specific condition, the controller 111 selects the type and processing conditions of the post-processing and performs the selected post-processing on the currently captured image. The specific condition is a specific imaging part, a specific case, a respiratory cycle, or the like.

For example, when the imaging part of the currently captured image is a side view of the knee joint, the controller 111 performs position adjustment on the currently captured image so that the articular surfaces of the inner condyle and the outer condyle of the femur are positioned at the center of the image.

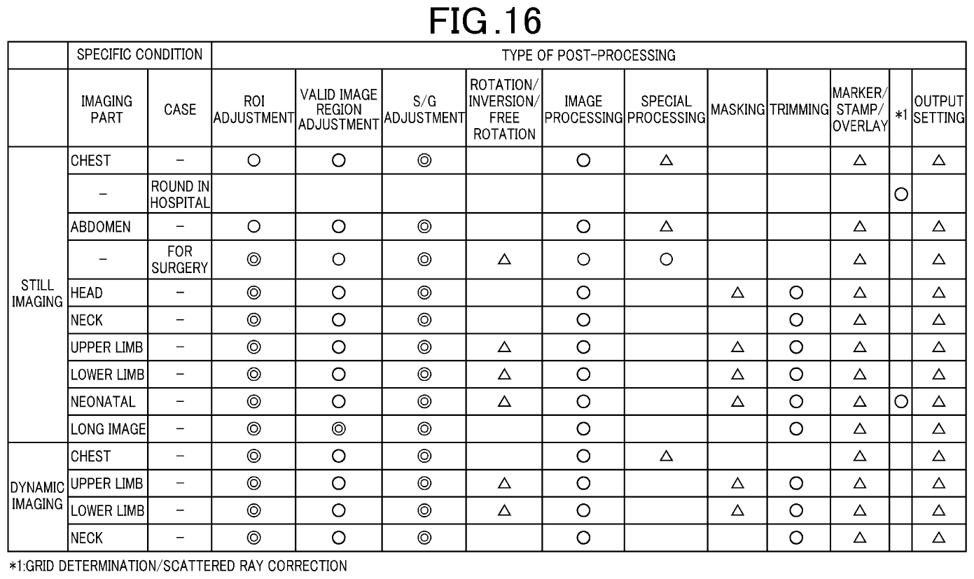

For example, FIG. 16 illustrates an example of the types of post-processing performed by the controller 111 for respective specific imaging parts and respective specific cases.

In the example illustrated in FIG. 16, the priorities of post-processing are indicated by ⊚ (double circle), ○ (circle), and Δ (triangle) in the descending order. The blanks indicate that processing is not performed.

The controller 111 selects the type and processing conditions of post-processing on the basis of a reference image obtained by imaging the same imaging part as the current imaging part and performs the selected post-processing on the currently captured image.

The reference image is an image that is suitable for a clinician or a radiologist. The image suitable for a clinician or a radiologist is an image that the clinician or the radiologist prefer for diagnosis or an image that makes diagnosis easier.

Specifically, the controller 111 extracts the difference between the currently captured image and the reference image and performs post-processing on the currently captured image so as to eliminate the difference. This processing is performed based on machine learning using standard images as teacher data.

The controller 111 selects the type and processing conditions of post-processing based on external information and performs the selected post-processing on the currently captured image.

For example, a case will be described where the X-ray imaging system 1 comprises an optical camera (not illustrated) that captures an optical image of an area including at least a part of an area irradiated with X-rays from the X-ray tube device 25. In this case, the controller 111 determines the imaging direction of the currently captured image based on an optical image as the external information and performs processing of adding an annotation indicating the determined imaging direction to the currently captured image. In particular, the controller 111 may be configured to perform the processing of adding an imaging direction as an annotation when the imaging part of the currently captured image is the head or the neck.

The controller 111 selects the type and processing conditions of post-processing based on statistical information and performs the selected post-processing on the currently captured image. The statistical information is, for example, statistical information on imaging failure reasons for previously captured images or quality assurance (QA) rejection reasons for previously captured images.

A specific case will be described where the QA rejection reason of the previous image of the same imaging part as the imaging part of the currently captured image is mismatch of the trimming size. In this case, the controller 111 adjusts the output trimming size of the currently captured image so that the trimming sizes match.

A case will be described in which the QA rejection reason of the previously captured images is that a catheter tip enhanced image or a gauze enhanced image is not added at the time of the outputting the image. In this case, the controller 111 generates a catheter tip enhanced image or a gauze enhanced image of the currently captured image. The controller 111 adds the generated catheter tip enhanced image or gauze enhanced image to the currently captured image and outputs the images.

A case will be described where the X-ray imaging device 10 and the X-ray generation device 20 are not coordinated with each other. In this case, the controller 111 applies the same processing as the image processing applied to the previously captured images to the currently captured image to which image processing based on the irradiation conditions and grid information at the time of imaging has not yet been applied. For example, if the grid removal processing is applied at a statistically high rate in the previously captured images, the controller 111 applies the grid removal processing to the currently captured image. The controller 111 may display a dialog for confirming whether or not to apply the grid removal processing to the currently captured image at the time of outputting the currently captured image in addition to applying the grid removal processing to the currently captured image.

A case will be described where the examination end button A8 is pressed by the user. In this case, the controller 111 may display a dialog in the examination screen 113c to ask whether or not the trimming size for outputting the currently captured image should be adjusted to the trimming size that is applied statistically at a high rate to the images captured in the past.

The controller 111 may select the type and processing conditions of the post-processing based on the statistical information aggregated by the statistical aggregation function of the X-ray generation console 22 and perform the selected post-processing on the currently captured image.

For example, the controller 111 may determine whether or not to automatically perform the selected post-processing on the user basis based on the number of times of image failures of the user as the statistical information.

For example, the controller 111 may determine whether or not to perform post-processing corresponding to the imaging failure reason on the currently captured image based on the imaging failure reason as statistical information.

For example, the controller 111 may perform adjustment such that the S value, the G value, the EI value, the TI value, and the like of the currently captured image become the same as the S value, the G value, the EI value, the TI value, and the like as the statistical information in past images. The past images are images obtained by imaging the same imaging part as the imaging part of the currently captured image.

For example, the controller 111 may select the type and processing conditions of post-processing on the basis of the frequency of use of the FPD 12, the X-ray generation device 20, the grid, and the like as the statistical information. Thereafter, the controller 111 may determine whether or not to perform the selected post-processing on the currently captured image. To be specific, the controller 111 selects the type and processing conditions of post-processing for correcting the influence of the deterioration of the FPD 12, the X-ray generation device 20, the grid, and the like.

The statistical information may be statistical information based on an input operation by the user or statistical information aggregated from an operation log or the like, and the statistical information may be accumulated by any method. Among these items of statistical information, the items for selecting the type and processing conditions of post-processing may be selected in advance as setting for each facility, each console device, each user, each imaging room, or the like. Among these items of statistical information, a selection policy for each item as to whether or not to perform the selected post-processing may be preset for each facility, each console device, each user, each imaging room, or the like. The priority of the items of the statistical information used for selecting the type and processing conditions of the post-processing may also be specifiable for each facility, each apparatus, or each department.

The controller 111 may select the type and processing conditions of post-processing by the above-described predetermined methods and perform the selected post-processing on the currently captured image in response to an input operation by the user.

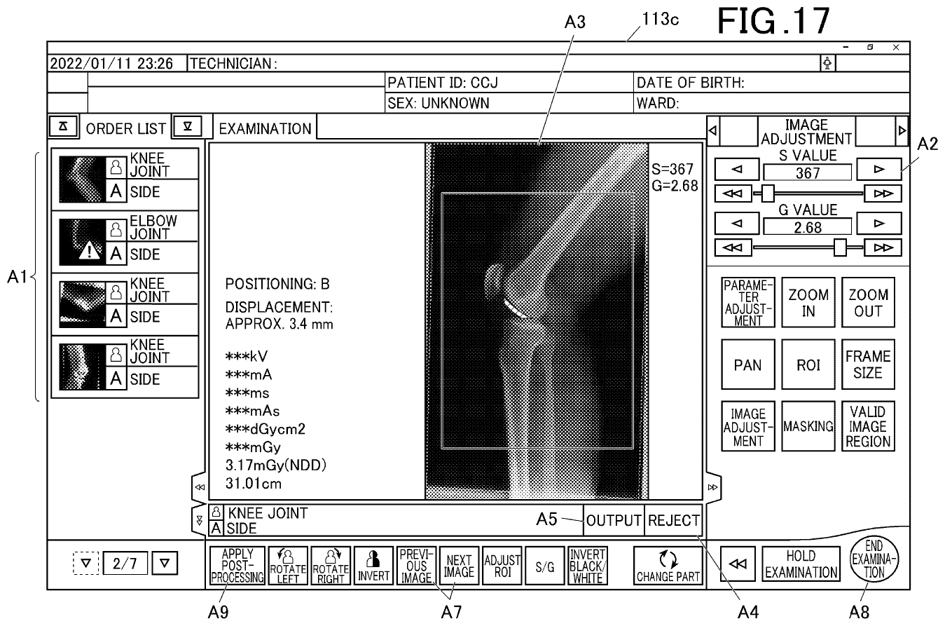

FIG. 17 illustrates an example of the examination screen 113c displayed on the display part 113.

The controller 111 creates, on the examination screen 113c, an imaging selection button A1, a setting area A2, an image display area A3, an image failure button A4, an outputting button A5, a switching button A7, an examination end button A8, a post-processing application button A9 for an instruction to execute post-processing, and the like.

Concretely, the controller 111 receives a user instruction to perform the selected post-processing on the currently captured image through the post-processing application button A9. When the user determines that the post-processing is to be performed on the currently captured image, the user presses the post-processing application button A9. In response to the post-processing application button A9 being pressed, the controller 111 performs the selected post-processing on the currently captured image. The selected post-processing performed by the controller 111 in response to the post-processing application button A9 being pressed may be a plurality of types of post-processing or a plurality of times of post-processing. Whether or not to perform the selected post-processing on the currently captured image may be set in advance by the user.

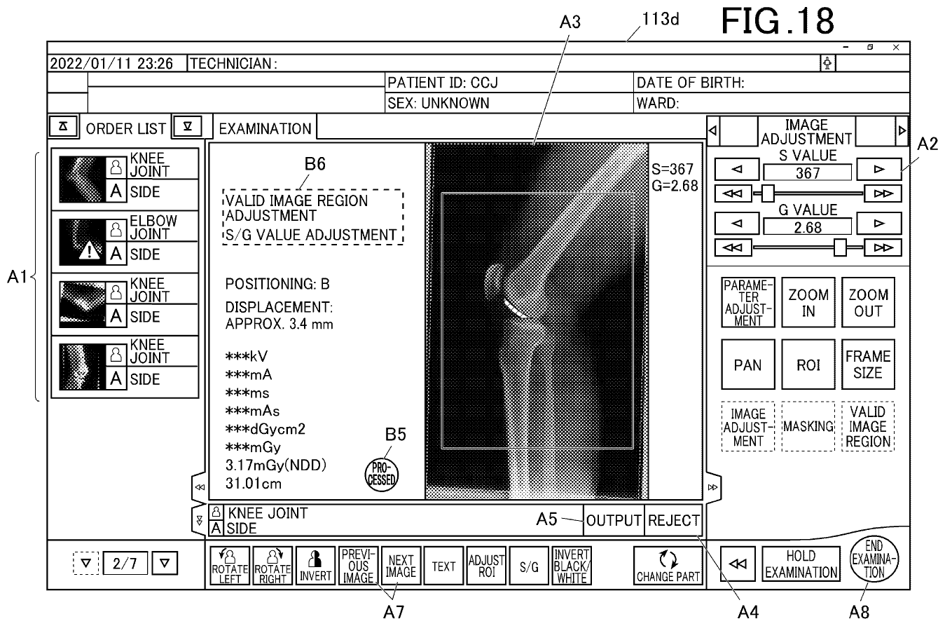

Next, the controller 111 performs display control on the examination screen 113d (see FIG. 18) so that the image on which the post-processing has been performed in step S21 and the image on which the post-processing has not been performed can be distinguished from each other (step S22). Thereafter, the controller 111 ends the processing. That is, the controller 111 displays, on the display part 113, the radiographic image on which the post-processing (predetermined processing) has been executed by the processing section. The controller 111 functions as a display controller.

Specifically, the controller 111 adds an identification mark or the like to the image on which the post-processing has been performed.

For example, as illustrated in FIG. 18, the controller 111 adds a post-processing done mark B5 as an identification mark to the image on which the post-processing has been performed.

The controller 111 may add an identification mark or the like indicating that the post-processing has not been performed to the image on which the post-processing has not been performed.

The controller 111 may add a character or a symbol to the image instead of the identification mark.

The controller 111 may display the identification mark or the identification character or symbol on any of the imaging selection button A1, a thumbnail in the imaging selection button A1, and a dialog instead of on the image. A case will be described in which one imaging corresponding to one imaging selection button A1 includes a plurality of images and the post-processing has been performed on at least one of the plurality of images. In this case, the controller 111 adds an identification mark or an identification character or symbol to the imaging selection button A1.

The controller 111 may change the color, shape or the like of the imaging selection buttons A1 so that the user can distinguish between an image on which post-processing has been performed and an image on which post-processing has not been performed.

As illustrated in FIG. 18, the controller 111 may display, in the examination screen 113d, the contents B6 of the post-processing (the type of post-processing) performed on the currently captured image in the step S21.

The controller 111 performs display control for the examination screen 113d so that the user cannot additionally perform the post-processing that has been performed on the currently captured image in step S21.

For example, as illustrated in FIG. 18, the controller 111 grays out (shown as broken lines in FIG. 18) some of the buttons in the setting area A2 so that they cannot be selected. The controller 111 may not display, on the examination screen 113d, the button for selecting the post-processing that has been performed on the currently captured image.

For example, when the currently captured image is used for surgery, the controller 111 displays the catheter tip enhanced image or the gauze enhanced image of the currently captured image on the examination screen 113d.

For example, the controller 111 displays the catheter tip enhanced image or the gauze enhanced image and the original image thereof side by side on the examination screen 113d.

The controller 111 displays catheter tip enhanced images and gauze enhanced images with different enhancement levels side by side on the examination screen 113d.

For example, when the currently captured image is used for surgery, the controller 111 displays an image captured before surgery on the examination screen 113d.

As described above, the imaging control device 11 (control device) includes the first acquirer (controller 111) that acquires a radiographic image, the processing section (controller 111) that executes predetermined processing (post-processing) on the radiographic image acquired by the first acquirer, the second acquirer (controller 111) that acquires imaging conditions of the radiographic image, and the selection section (controller 111) that automatically selects the type and processing conditions of predetermined processing to be executed by the processing section based on the imaging conditions acquired by the second acquirer.

Therefore, since appropriate post-processing based on the imaging conditions can be automatically executed, it is possible to further reduce the work load of executing predetermined processing (post-processing) on the radiographic image.

In the imaging control device 11, the selection section automatically determines the type and processing conditions of the predetermined processing based on any of machine learning, statistical information, and the contents of the predetermined processing executed on a radiographic image captured in the past.

Therefore, it is possible to execute more appropriate post-processing based on any of machine learning, statistical information, and the contents of the predetermined processing executed on a radiographic image captured in the past.

The imaging control device 11 comprises a display controller (controller 111) that displays, on the display part 113, the radiographic image on which the predetermined processing has been executed by the processing section.

Therefore, the user can check the radiographic image on which the post-processing has been automatically executed.

In the imaging control device 11, the radiographic image includes a plurality of frame images.

Accordingly, appropriate post-processing based on the imaging conditions can be automatically executed even in dynamic imaging. Therefore, it is possible to further reduce the workload of executing predetermined processing (post-processing) on the radiographic image.

Although the embodiment of the present invention has been described, the scope of the present invention is not limited to the above-described embodiments but includes the scope of the invention described in the claims and its equivalent thereof.

For example, although the X-ray imaging system 1 is installed in an imaging room in the above embodiments, the present invention is not limited thereto. The X-ray imaging system 1 may be configured as a mobile medical car.

For example, the controller 111 may calculate statistical information associated with execution of the post-processing and cause the display part 113 to display the statistical information or output the statistical information to an external device. The controller 111 calculates, as the statistical information, the ratio of cases requiring the post-processing with respect to each examination, the time from when post-processing is determined necessary to when the post-processing is performed, or the number of images on which the post-processing is performed although the post-processing is not determined necessary. The controller 111 calculates the statistical information based on information on determination of the necessity of post-processing stored in the storage section 112 and its associated information (determination target, determination date and time), and the post-processing execution result after determining whether or not post-processing is necessary (execution status, execution date and time). Thus, it is possible to quantitatively know whether or not the post-processing is efficiently performed, and it is possible to share the necessity of post-processing depending on a part or a condition, a caution in performing the post-processing, and the like.

For example, the X-ray imaging system 1 may include a display terminal device (not illustrated) as another display part in addition to the display part 113. The display terminal device is, e.g., a mobile terminal or the like. In this case, only the radiographic image determined to require post-processing may be limitedly displayed on the display terminal device as a security measure when the mobile terminal is taken out to the outside or in order to work efficiently with a display area smaller than the display part 113.

The display terminal device may have a function as the processing section, and the same contents may be displayed on the display part 113 and the display terminal device. This allows sharing the information required for post-processing with each other and sharing the work. For example, only the necessity of post-processing is determined in the imaging control device 11 (controller 111). The post-processing on a radiographic image requiring the post-processing is performed on the display terminal device.

The determination on the necessity of post-processing and the selection of the type and processing conditions of the post-processing may not be performed in the imaging control device 11 (controller 111) but in the display terminal device. Alternatively, the controller 111 associates the determination result of whether or not post-processing is necessary and the information on the selected type and processing conditions of the post-processing with the captured image and transmits the determination result and the information to another control device. Another user may perform post-processing in the other control device under an environment different from that of the X-ray imaging system 1. This allows workload distribution by transferring post-processing to another person in a case of a new radiographer, during manpower shortage hours, or the like.

Although some embodiments of the present invention have been described, the scope of the present invention is not limited to the above-described embodiments and includes the scope of the invention described in the claims and its equivalents.

Although embodiments of the present invention have been described and illustrated in detail, the disclosed embodiments are made for purposes of illustration and example only and not limitation. The scope of the present invention should be interpreted by terms of the appended claims.

1. A control device, comprising:
a hardware processor that acquires a radiographic image, wherein
the hardware processor acquires an imaging condition of the radiographic image, automatically selects a type and a processing condition of predetermined processing to be executed on the acquired radiographic image based on the acquired imaging condition and executes the selected predetermined processing on the radiographic image.

2. The control device according to claim 1, wherein the hardware processor automatically selects the type and the processing condition of the predetermined processing based on any one of machine learning, statistical information, and a content of the predetermined processing executed on a radiographic image captured in a past.

3. The control device according to claim 1, wherein the hardware processor displays, on a display part, the radiographic image on which the predetermined processing has been executed.

4. The control device according to claim 1, wherein the radiographic image includes a plurality of frame images.

5. A control method, comprising:
acquiring a radiographic image;
acquiring an imaging condition of the radiographic image;
automatically selecting a type and a processing condition of predetermined processing based on the acquired imaging condition; and
executing the predetermined processing on the acquired radiographic image.

6. A non-transitory computer-readable recording medium that causes a computer of a control device to function as:
a hardware processor that acquires a radiographic image, wherein
the hardware processor acquires an imaging condition of the radiographic image, automatically selects a type and a processing condition of predetermined processing to be executed on the acquired radiographic image based on the acquired imaging condition and executes the selected predetermined processing on the radiographic image.
